(19) United States
(12) Patent Application Publication
(10) Pub. No.: US 2016/0039759 A1
(43) Pub. Date: Feb. 11, 2016

(54) PROCESS FOR THE PREPARATION OF PERAMPANEL

(71) Applicant: CADILA HEALTHCARE LIMITED, Gujarat (IN)

(72) Inventors: Sanjay Jagdish DESAI, Gujarat (IN); Jayprakash Ajitsingh Parihar, Gujarat (IN); Kuldeep Natwarlal Jain, Gujarat (IN); Sachin Ashokrao Patil, Gujarat (IN)

(21) Appl. No.: 14/816,861

(22) Filed: Aug. 3, 2015

(30) Foreign Application Priority Data
Aug. 5, 2014 (IN) 2512/MUM/2014

(51) Int. Cl. C07D 213/64

(52) U.S. Cl. CPC C07D 213/64

(57) ABSTRACT

The present invention relates to processes for the preparation of perampanel and its intermediates.

### FIELD OF THE INVENTION

The field of the invention relates to processes for the preparation of perampanel. In particular, the present invention relates to a process for the preparation of perampanel and intermediates thereof.

### BACKGROUND OF THE INVENTION

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

Perampanel, a non-competitive AMPA receptor antagonist, is the active ingredient of FYCOMPA® tablets (U.S) which is approved as an adjunctive therapy for the treatment of partial on-set seizures with or without secondarily generalized seizures in patients with aged 12 years and older. Chemically, Perampanel is 5′-(2-cyanophenyl)-1′-phenyl-2,3′-bipyridinyl-6′(1′H)-one, with an empirical formula C23H15N30 and molecular weight 349.384 g/mol which is represented by Formula (I).

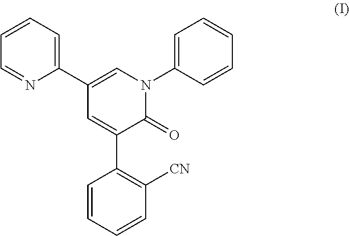 [SMILES: N#Cc1ccccc1-c1cc(-c2ccccn2)cn(-c2ccccc2)c1=O]

U.S. Pat. No. 6,949,571 B2 discloses perampanel and its various processes for preparation thereof.

U.S. Pat. No. 7,759,367 B2 discloses the pharmaceutical composition of perampanel and an immunoregulatory agent and their uses.

U.S. Pat. No. 8,304,548 B2 discloses the reaction of 5′-bromo-1′-phenyl-[2,3′-bipyridin]-6′(1′H)-one with 2-(1,3,2-dioxaborinan-yl)benzonitrile in the presence of palladium compound, a copper compound, a phosphorus compound and a base to form perampanel of Formula (I). Also discloses the crystalline hydrate, anhydrous crystal Form I, anhydrous crystal Form III, & anhydrous crystal Form V of perampanel of Formula (I).

U.S. Pat. No. 7,803,818 B2 discloses an amorphous form of perampanel. U.S. Pat. No. 7,718,807 B2 discloses salts of perampanel. International (PCT) publication No. WO 2013/102897 A1 discloses anhydrous crystalline Form III, V & VII of perampanel.

U.S. PG-Pub. No. 2013/109862 A1 discloses the method for preparing 2-alkoxy-5-(pyridin-2-yl)pyridine, which is an intermediate for preparing perampanel key starting material 5-(2′-pyridyl)-2-pyridone.

U.S. Pat. No. 7,524,967 B2 discloses the preparation of 5-(2′-pyridyl)-2-pyridone, an intermediate in the preparation perampanel.

International (PCT) publication No. WO 2014/023576 A1 discloses the preparation of cyanophenyl boronic acid, an intermediate in the preparation perampanel.

The prior-art processes suffer with problems of poor yield and requirement of chromatographic purification or series of crystallizations which further reduces the overall yield of the final product, which is overcome by the process of the present invention.

### SUMMARY OF THE INVENTION

In one general aspect, there is provided a process for the preparation of perampanel, the process comprising:

(a) reacting 5-(2-pyridyl)-1,2-dihydropyridin-2-one with a brominating agent to obtain 3-bromo-5-(2-pyridyl)- 1 ,2-dihydropyridin-2-one;

(b) reacting the 3-bromo-5-(2-pyridyl)-1,2-dihydropyridin-2-one with phenyl boronic acid to obtain 3-bromo-5-(2-pyridyl)-1-phenyl-1,2-dihydropyridine-2-one; and

(c) reacting the 3-bromo-5-(2-pyridyl)-1-phenyl-1,2-dihydropyridine-2-one with 2-(2-cyanophenyl)-1,3,2-dioxaborinate to obtain perampanel.

In another general aspect, there is provided a process for the preparation of 5-(2-pyridyl)-1,2-dihydropyridin-2-one comprising reacting 5-bromo-2-methoxy-pyridine with pyridin-2-yl boronic acid in the presence of palladium catalyst and a base to obtain 2-methoxy-5-(pyridin-2-yl)pyridine followed by acid-catalysed demethylation to obtain 5-(2-pyridyl)-1,2-dihydropyridin-2-one.

In another general aspect, there is provided a process for the preparation of 5-(2-pyridyl)-1,2-dihydropyridin-2-one comprising reacting 2-bromopyridine with 6-methoxypyridin-3-yl-3-boronic acid in the presence of palladium catalyst and a base to obtain 2-methoxy-5-(pyridin-2-yl) pyridine followed by acid-catalysed demethylation to obtain 5-(2-pyridyl)-1,2-dihydropyridin-2-one.

In another general aspect, there is provided a pharmaceutical composition comprising perampanel and one or more pharmaceutically acceptable carriers, excipients or diluents.

In another general aspect, there is provided a process for the preparation of 5-(2-pyridyl)-1,2-dihydropyridin-2-one substantially same as that shown in Scheme-1.

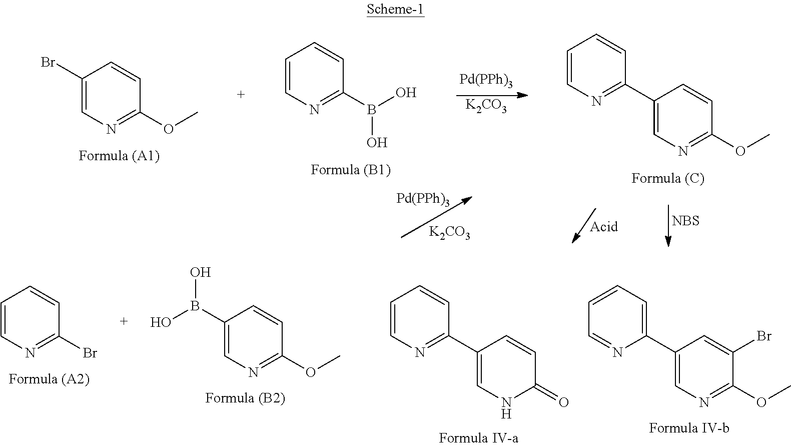

In another general aspect, there is provided a process for the preparation of perampanel as depicted in Scheme-2

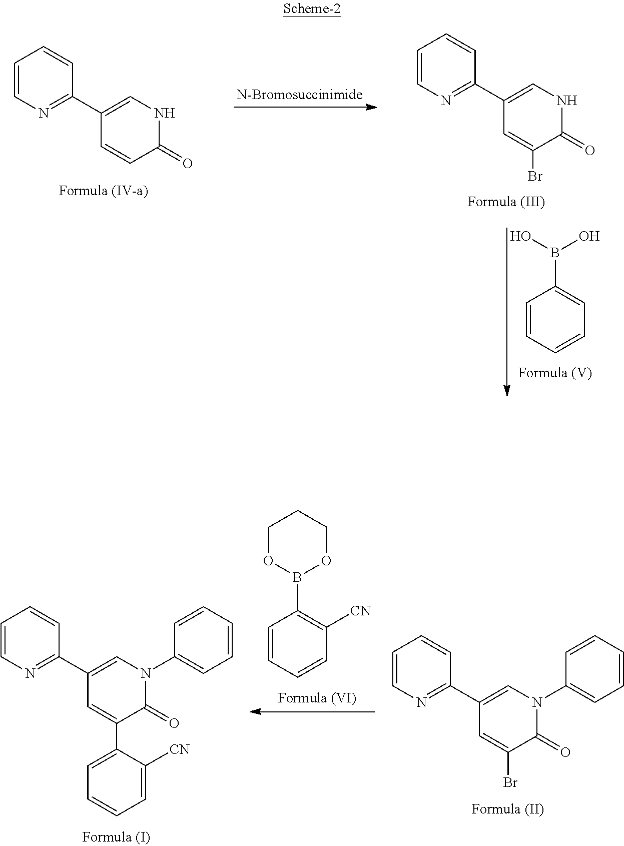

### DETAILED DESCRIPTION OF THE INVENTION

The above mentioned general and further specific aspects of the present invention are fulfilled by the description of the invention provided herein after.

All ranges recited herein include the endpoints, including those that recite a range “between” two values. This includes, at very least, the degree of expected experimental error, technique error and instrument error for a given technique used to measure a value.

In general, the term “obtaining” means removal of the solvent medium to obtain the product. Herein the removal of the solvent may be done by a technique which includes, for example, filtration, filtration under vacuum, decantation, centrifugation, distillation and distillation under vacuum.

The product obtained may further or additionally be dried to achieve the desired level of moisture and/or residual solvents. For example, the product may be dried in a hot air oven, tray drier, dried under vacuum and/or in a Fluid Bed Drier. The product may also be purified further by optional and additional purification(s) and isolation(s) to achieve level of purity and/or to obtain desired polymorphic form.

In general, the term isolation and purification includes operations like extraction, evaporation, crystallization, filtration, recrystallization or chromatography.

In one general aspect, there is provided a process for the preparation of perampanel, the process comprising:

(a) reacting 5-(2-pyridyl)-1,2-dihydropyridin-2-one with a brominating agent to obtain 3-bromo-5-(2-pyridyl)-1,2-dihydropyridin-2-one;

(b) reacting the 3-bromo-5-(2-pyridyl)-1,2-dihydropyridin-2-one with phenyl boronic acid to obtain 3-bromo-5-(2-pyridyl)-1-phenyl-1,2-dihydropyridine-2-one; and

(c) reacting the 3-bromo-5-(2-pyridyl)-1-phenyl-1,2-dihydropyridine-2-one with 2-(2-cyanophenyl)-1,3,2-dioxaborinate to obtain perampanel.

In general, the brominating agent is selected from one or more of N-bromosuccinimide (NBS), tribromoisocyanuric acid, acetic acid-bromine mixture, and liquid bromine In particular, the brominating agent is NBS.

In particular, the reaction of 5-(2-pyridyl)-1,2-dihydropyridin-2-one with NBS in N,N-dimethylformamide yields 3-bromo-5-(2-pyridyl)-1,2-dihydropyridin-2-one. The process of the present invention provides 3-bromo-5-(2-pyridyl)-1,2-dihydropyridin-2-one, which is obtained in 85% or more yield and having about 95% or more purity; particularly in 90% or more yield and about 98% or more purity; more particularly in 92% or more yield and about 99% or more purity; further more particularly in 92.5% or more yield and about 99.5% or more purity as measured by area percentage of HPLC.

The compound 3-bromo-5-(2-pyridyl)-1-phenyl-1,2-dihydropyridine-2-one is obtained by reacting 3-bromo-5-(2-pyridyl)-1,2-dihydropyridin-2-one and phenyl boronic acid in the presence of a copper salt and a base.

In general, the copper salt is selected from copper acetate, copper bromide, and copper iodide. The ligands may be used to promote copper-catalysed C-N coupling reactions. The ligands are selected from one or more of diamines, 1,10-phenanthroline, amino acids, N,N-diethylsalicylamide, ethylene glycol, 8-hydroxyquinoline, an aminoarenethiol, 1,1,1-tris(hydroxymethyl)ethane, 2-amino pyrimidine-4,6-diol, 1,1′-binaphthyl-2,2′-diol, ethyl 2-oxocyclo-hexane carboxylate. In particular, the ligands used for copper catalysed C-N coupling reaction is selected from 1,3-bis(2,4,6-trimethylphenyl)imidazolium chloride, 1,3-bis(2,6-diisopropylphenyl)imidazolium chloride, 1,3-bis(adamant-1-yl) imidazolium chloride, 1,3-bis(2,4,6-trimethylphenyl)-4,5-dihydroimidazolium tetrafluoroborate, 1,3-bis(2,6-diisopropylphenyl)imidazolidinium tetrafluoro-borate, 1,3-bis(2,4,6-trimethylphenyl)imidazolidinium chloride, and 1,3-bis(2,6-diisopropylphenyl)imidazolidinium chloride. In particular, the copper salt is selected from copper acetate or di-p-hydroxobis[(N,N,N′,N′-tetramethylethylene-diamine)copper(II)]chloride.

In general, the base is selected from an inorganic base or an organic base. The inorganic base is selected from sodium hydroxide, potassium hydroxide, lithium hydroxide, sodium carbonate, potassium carbonate, sodium bicarbonate, potassium bicarbonate, sodium hydride, potassium tert-butoxide, cesium carbonate, methyl lithium, butyl lithium, sodium amide, a dialkyl lithium amide; and the organic bases is selected from triethylamine, diisopropyl ethyl amine, diethylamine, N-methyl morpholine, pyridine, piperidine, and DBU. In particular, the base is pyridine.

In general, the compound 3-bromo-5-(2-pyridyl)-1,2-dihydropyridin-2-one is reacted with phenyl boronic acid in the presence of copper acetate and pyridine in N,N-dimethylformamide to obtain 3-bromo-5-(2-pyridyl)-1-phenyl-1,2-dihydro-pyridine-2-one. The process of the present invention obtained 3-bromo-5-(2-pyridyl)-1-phenyl-1,2-dihydro-pyridine-2-one in 90% or more yield and having about 95% or more purity; particularly in 92% or more yield and about 98% or more purity; more particularly in 95% or more yield and about 99% or more purity; further more particularly in 96% or more yield and about 99.5% or more purity as measured by area percentage of HPLC.

The compound 3-bromo-5-(2-pyridyl)-1-phenyl-1,2-dihydropyridine-2-one is reacted with 2-(2-cyanophenyl)-1,3,2-dioxaborinate in the presence of a palladium catalyst to obtain perampanel.

In general, the palladium catalyst may be formed in-situ by using palladium precursors and a necessary ligand or may be introduced as preformed palladium catalyst.

The palladium precursor is selected from allylpalladium chloride dimer, bis (acetonitrile)palladium(II) chloride, bis(benzonitrile)palladium(II) chloride, bis (dibenzylideneacetone)palladium, palladium(II)acetate, palladium(II) bromide, palladium (II) chloride, palladium(II) hydroxide, palladium(II) trifluoroacetate, tetrakis(acetonitrile)palladium(II)tetrafluoroborate, tris(dibenzylideneacetone)-dipalladium(0), and tris(dibenzylideneacetone)dipalladium chloroform adduct; and the ligand is selected from triphenyl phosphine, tri(2-furyl) phosphine, tri-o-tolylphosphine, trimesitylphosphine, tricyclohexylphosphine, triisopropyl-phosphine, tri-n-butylphosphine, di-tert-butylmethylphosphine and tri-tert-butylphosphine.

The preformed palladuim catalyst is a palladium/ligand complex selected from [1,2-bis(diphenylphosphino)ethane]dichloropalladium(II), 1,1′-bis(diphenyl-phosphino) ferrocenepalladium(II)dichloride dichloromethane adduct, bis(tricy-clohexylphosphine)palladium(0), bis(triethylphosphine)-palladium(II) chloride, bis(triphenylphosphine)palladium(II) acetate, bis(triphenylphosphine)palladium(II) chloride, Bis(tri-t-butylphosphine)palladium(0), bis[1,2-bis(diphenylpho-sphino)ethane]palladium(0), bis[tri(o-tolyl)phosphine]palladium(II)chloride, Dichlorobis-(tricyclohexylphosphine) palladium(II), tetrakis(triphenylphosphine)-palladium(0) and transBenzyl(chloro)bis(triphenylphosphine) palladium(II); preferably the palladium catalyst is tetrakis(triphenylphosphine)-palladium(0).

In general, a base is used in addition to the palladium catalyst. The base may be selected from organic bases or inorganic bases described herein above.

In general, the compound 3-bromo-5-(2-pyridyl)-1-phenyl-1,2-dihydropyridine-2-one is reacted with 2-(2-cyanophenyl)-1,3,2-dioxaborinate in the presence of tetrakis(tri-phenylphosphine)palladium and potassium carbonate in N,N-dimethylformamide to obtain perampanel.

In general, the palladium catalyst or the catalytic precursor and ligand may be used in an amount 0.001 mole to 0.5 moles per mole of 3-bromo-5-(2-pyridyl)-1-phenyl-1,2-dihydropyridine-2-one.

The reported prior-art processes for preparation of 3-bromo-5-(2-pyridyl)-1-phenyl-1,2-dihydropyridine-2-one involves firstly the coupling of 5-(2-pyridyl)-1,2-dihydropyridin-2-one with phenyl boronic acid followed by bromination of the resulted product. This process sequence results in about 50-64% overall yields of 3-bromo-5-(2-pyridyl)-1-phenyl-1,2-dihydropyridine-2-one having the purity of about 80-90%, as measured by area percentage of HPLC. In comparison, the process of the present invention provides 3-bromo-5-(2-pyridyl)-1-phenyl-1,2-dihydropyridine-2-one in an overall yields of 80-90% with about 99.5% or more purity, as measured by area percentage of HPLC. This results in the preparation of perampanel with high purity and which requires less purification steps. Thus, the present process provides higher overall yields of perampanel due to higher yields of 3-bromo-5-(2-pyridyl)-1-phenyl-1,2-dihydropyridine-2-one.

In another general aspect, there is provided perampanel prepared by using 3-bromo-5-(2-pyridyl)-1,2-dihydropyridin-2-one prepared by the process of present invention, having a purity of about 99% or more, particularly of about 99.5% or more, more particularly of about 99.8% or more, still more particularly of about 99.9% or more as measured by area percentage of HPLC.

In another general aspect, there is provided a process for the preparation of 3-bromo-5-(2-pyridyl)-1-phenyl-1,2-dihydropyridine-2-one, the process comprising reacting 5-(2-pyridyl)-1,2-dihydropyridin-2-one with a brominating agent to obtain 3-bromo-5-(2-pyridyl)-1,2-dihydropyridin-2-one and reacting the 3-bromo-5-(2-pyridyl)-1,2-dihydropyridin-2-one with phenyl boronic acid to obtain 3-bromo-5-(2-pyridyl)-1-phenyl-1,2-dihydropyridine-2-one.

In another general aspect, there is provided a process for the preparation of 5-(2-pyridyl)-1,2-dihydropyridin-2-one and 5′-bromo-6′-methoxy-2,3′-bipyridine as described and depicted in Scheme-1, herein earlier.

In another general aspect, there is provided a process for the preparation of 5-(2-pyridyl)-1,2-dihydropyridin-2-one comprising reacting 5-bromo-2-methoxypyridine with pyridin-2-yl boronic acid in the presence of palladium catalyst and a base to obtain 2-methoxy-5-(pyridin-2-yl)pyridine followed by acid-catalysed demethylation to obtain 5-(2-pyridyl)-1,2-dihydropyridin-2-one.

In another general aspect, there is provided a process for the preparation of 5-(2-pyridyl)-1,2-dihydropyridin-2-one comprising reacting 2-bromopyridine with 6-methoxypyridin-3-yl-3-boronic acid in the presence of palladium catalyst and a base to obtain 2-methoxy-5-(pyridin-2-yl) pyridine followed by acid-catalysed demethylation to obtain 5-(2-pyridyl)-1,2-dihydropyridin-2-one.

In general, the demethylation is carried out in presence of an acid. The acid comprises one or more of mineral acid selected from hydrofluoric acid, hydrobromic acid, hydroiodic acid, hydrochloric acid, nitric acid, carbonic acid and sulphuric acid; alkylsulfonic acid selected from methane sulfonic acid, ethanesulfonic acid, and trifluoroethanesulfonic acid; arylsulfonic acid selected from benzenesulphonic acid and p-toluenesulfonic acid; organic acid selected from acetic acid, propionic acid, butyric acid, fumaric acid, tartaric acid, oxalic acid, malonic acid, maleic acid, inane acid, succinic acid, benzoic acid, mandelic acid, ascorbic acid, lactic acid, gluconic acid and citric acid. In particular, the hydrochloric acid is used for demethylation.

In general, the solvents for the reactions of the present invention is selected from the group comprising water, alcohols, esters, ethers, ketones, chlorinated solvents, nitrile solvents or any combinations thereof The alcoholic solvent is selected from methanol, ethanol, isopropanol, n-propanol and butanol; the ester solvent is selected from ethyl acetate, methyl acetate, n-butyl acetate, isobutyl acetate, sec-butyl acetate and isopropyl acetate; the ether solvent is selected from diethyl ether, diisopropyl ether, methyl tert-butyl ether, tetrahydrofuran and 1,4-dioxane; the ketone solvent is selected from acetone, methylethylketone and methylisobutylketone; the chlorinated solvent is selected from chloroform, methylene chloride, ethylene dichloride and carbon tetrachloride; the nitrile solvent is acetonitrile.

In another general aspect, there is provided processes for the preparation of perampanel substantially as depicted in one or more of reaction Schemes 3-9 as herein below.

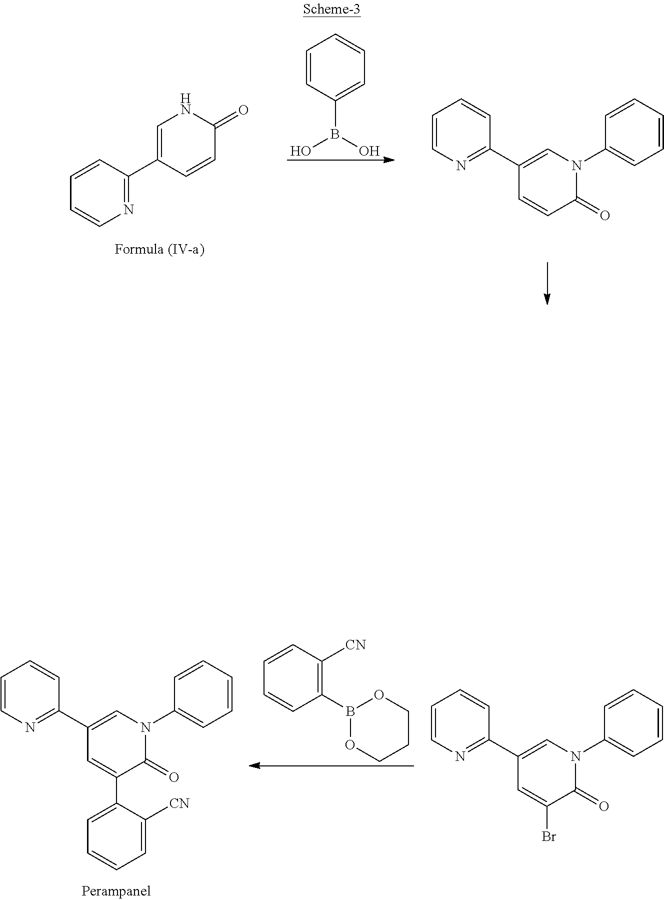

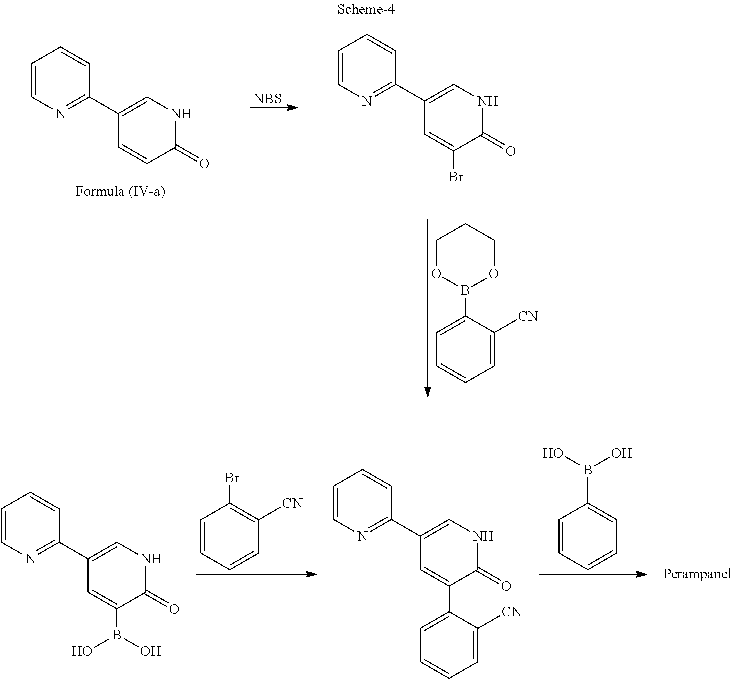

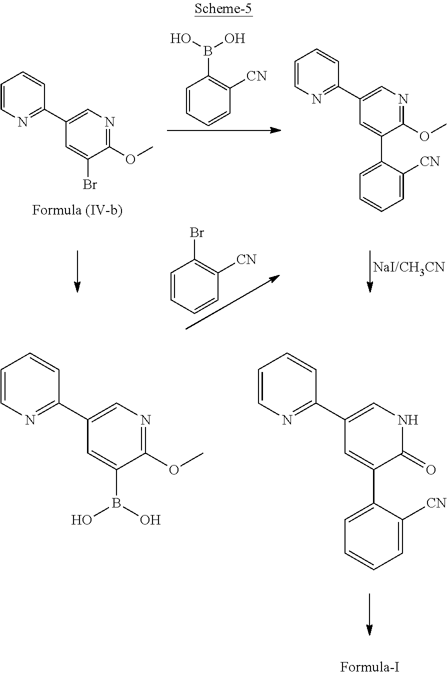 [SMILES: COc1ncc(-c2ccccn2)cc1Br ; N#Cc1ccccc1B(O)O ; COc1ncc(-c2ccccn2)cc1-c1ccccc1C#N ; N#Cc1ccccc1Br ; COc1ncc(-c2ccccn2)cc1B(O)O ; N#Cc1ccccc1-c1cc(-c2ccccn2)c[nH]c1=O]

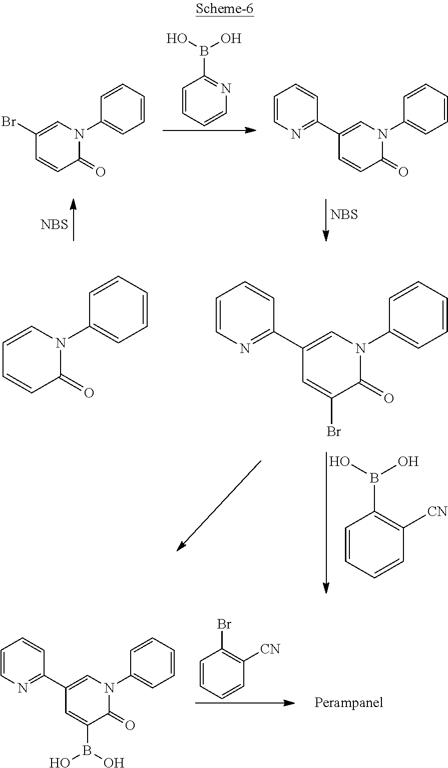

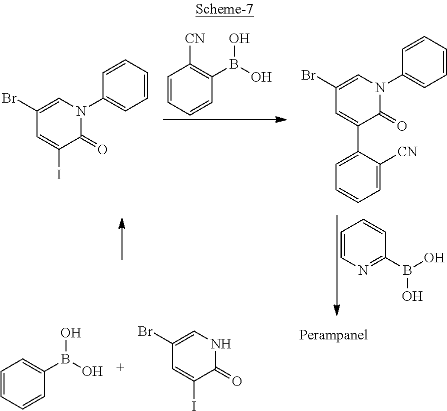

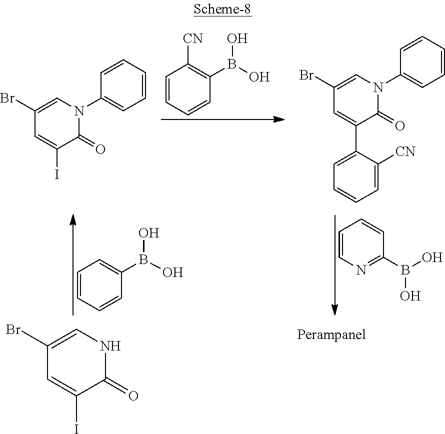

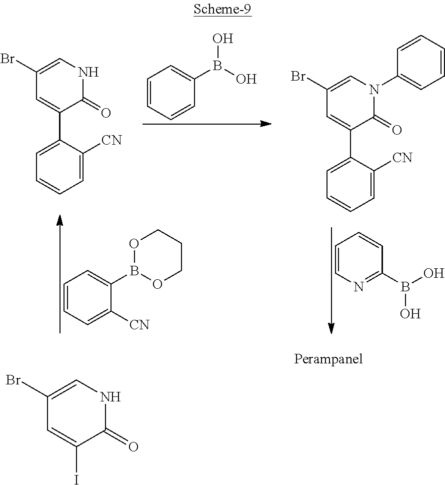

In another general aspect, there is provided a pharmaceutical composition comprising perampanel and one or more pharmaceutically acceptable carriers, excipients or diluents.

In another general aspect, there is provided perampanel having particle size distributions, D(10) of about 50 μm or less, D(50) of about 200 μm or less, and D(90) of about 400 μm or less; or D(10) of about 25 μm or less, D(50) of about 100 μm or less, D(90) of about 250 μm or less.

The invention also encompasses pharmaceutical compositions comprising perampanel of the present invention. As used herein, the term “pharmaceutical compositions” or “pharmaceutical Formulations” includes tablets, pills, powders, liquids, suspensions, emulsions, granules, capsules, suppositories, or injection preparations.

Pharmaceutical compositions containing the perampanel of the present invention may be prepared by using diluents or excipients such as fillers, bulking agents, binders, wetting agents, disintegrating agents, surface active agents, and lubricants. Various modes of administration of the pharmaceutical compositions of the invention can be selected depending on the therapeutic purpose, for example tablets, pills, powders, liquids, suspensions, emulsions, granules, capsules, suppositories, or injection preparations.

### EXAMPLES

The present invention is further illustrated by the following examples which is provided merely to be exemplary of the invention and do not limit the scope of the invention. Certain modification and equivalents will be apparent to those skilled in the art and are intended to be included within the scope of the present invention.

Example-A: Preparation of 5-(2-pyridyl)-1,2-dihydropyridin-2-one In a 500 mL round bottom flask, equipped with a mechanical stirrer, thermometer and an addition funnel, a solution of 188.80 g 5-bromo-2-methoxypyridine in 190 mL tetrahydrofuran and 12.92 g pyridine-2-yl boronic acid were added and refluxed. The reaction mixture was cooled to 25-30° C. and aqueous solution of hydrochloric acid was added and stirred for 1 hour. The reaction mixture was neutralized with aqueous sodium hydroxide and extracted with tetrahydrofuran.

The organic layer was washed with saline water, dried over anhydrous magnesium sulfate, and then evaporated to obtain the titled compound.

### Example-1

### Preparation of 3-bromo-5-(2-pyridyl)-1,2-dihydropyridin-2-one

In a 2 L round bottom flask, equipped with a mechanical stirrer, thermometer and an addition funnel, 201.5 g 5-(2-pyridyl)-1,2-dihydropyridin-2-one, 208.3 g N-bromosuccinimide and 1300 mL N,N-dimethylforamide were stirred at 25-30° C. for 2-3 hours. After completion of the reaction, the reaction mixture was poured into water and stirred for 30 min. The precipitate was filtered, washed with N,N-dimethylforamide and dried at 50° C. to obtain 230 g title compound.

### Example-2

### Preparation of 3-bromo-5-2-pyridyl)-1-phenyl-1,2-dihydropyridine-2-one

In a 500 mL round bottom flask, equipped with a mechanical stirrer, thermometer and an addition funnel, a solution of 18.75 g 3-bromo-5-(2-pyridyl)-1,2-dihydropyridin-2-one in 300 mL methylene dichloride, 18.36 g 1-phenyl boronic acid, 3.47 g palladium triphenylphosphine and 10 mL triethyl amine were added and the reaction mixture was stirred for 1 hour at 25-35° C. The reaction mixture was filtered and the filtrate was evaporated to dryness. The residue was crystallised from ethyl acetate to obtain the title compound.

### Example-3

### Preparation of Perampanel

In a 1 L round bottom flask, equipped with a mechanical stirrer, thermometer and an addition funnel, a suspension of 188 g 3-bromo-5-(2-pyridyl)-1-phenyl-1,2-dihydropyridine-2-one, 161.2 g 2-(1,3,2-dioxaborinan-2-yl)benzonitrile, 3.0 g tetrakis(triphenylphosphine)-palladium(0), 10 mL triethylamine (10 mL) in 300 mL methylene dichloride were stirred at 25-30° C. for 12 hours. To the reaction mixture was added 5 mL conc. aqueous ammonia, 10 mL water and 40 mL ethyl acetate. The separated organic layer was washed with water and saturated saline solution and dried over magnesium sulfate. The solvent was removed under vacuum. Ethyl acetate was added to the residue and heated obtain clear solution. n-hexane was added to this solution and cooled to 25-30° C. The obtained solid was filtered and washed with ethyl acetate and dried to obtain perampanel.

### Example-4

### Preparation of 3-Bromo-5-(2-pyridyl)-1,2-dihydropyridin-2-one

In a 2 L round bottom flask, equipped with a mechanical stirrer, thermometer and an addition funnel, 100 g 5-(2-pyridyl)-1,2-dihydropyridin-2-one, 108.5 g N-bromosuccinimide and 500 mL N,N-dimethylforamide were stirred at 30-35° C. for 3 hours. 100 mL water was added to the reaction mixture at 5-15° C. and stirred at 30-35° C. for 1 hour. The solid obtained was filtered, washed with water and dried to obtain 129 g 3-bromo-5-(2-pyridyl)-1,2-dihydropyridin-2-one.

### Example-5

### Preparation of 3-bromo-5-(2-pyridyl)-1-phenyl-1,2-dihydropyridine-2-one

In a 2 L round bottom flask, equipped with a mechanical stirrer, thermometer and an addition funnel, 100 g 3-bromo-5-(2-pyridyl)-1,2-dihydropyridin-2-one, 72.8 g phenylboronic acid and 500 mL N,N-dimethylformamide were added at 30-35° C. and stirred. 11.9 g copper acetate and 15.7 g pyridine were added and air was purged into the reaction mixture and stirred for 16 hours at 30-35° C. After the completion of the reaction, the reaction mixture was poured into 1200 mL aqueous ammonia at 10-15° C. and stirred for 2 hours at 30-35° C. The obtained solid was filtered, washed with water and dried to obtain 120 g 3-bromo-5-(2-pyridyl)-1-phenyl-1,2-dihydropyridine-2-one.

### Example-6

### Purification of 3-bromo-5-(2-pyridyl)-1-phenyl-1,2-dihydropyridine-2-one

In a 1 L round bottom flask, equipped with a mechanical stirrer, thermometer and an addition funnel, 100 g 3-bromo-5-(2-pyridyl)-1-phenyl-1,2-dihydropyridine-2-one and 500 mL isopropyl alcohol were stirred at 60-65° C. for 30 min. The reaction mixture was cooled to 20-25° C. and stirred for 30 min. The reaction mixture was filtered, washed with isopropanol and dried to obtain 96 g pure 3-bromo-5-(2-pyridyl)-1-phenyl-1,2-dihydropyridine-2-one.

### Example-7

### Preparation of Perampanel

In a 1 L round bottom flask, equipped with a mechanical stirrer, thermometer and an addition funnel, 100 g 3-bromo-5-(2-pyridyl)-1-phenyl-1,2-dihydropyridine-2-one and 125 g 2-(1,3,2-dioxaborinan-2-yl)benzonitrile and 1500 mL N,N-dimethylformamide were added under inert atmosphere. 44 g potassium carbonate and 4.2 g palladium tetrakis were added and stirred at 115-125° C. for 3 hours. The solvent was removed under vacuum. Ethyl acetate was added to the residue and the organic layer was distilled off to obtain perampanel (78 g).

While the present invention has been described in terms of its specific embodiments, certain modifications and equivalents will be apparent to those skilled in the art and are intended to be included within the scope of the present invention.

We claim:

1. A process for the preparation of perampanel, the process comprising:
(a) reacting 5-(2-pyridyl)-1,2-dihydropyridin-2-one with a brominating agent to obtain 3-bromo-5-(2-pyridyl)-1,2-dihydropyridin-2-one;
(b) reacting the 3-bromo-5-(2-pyridyl)-1,2-dihydropyridin-2-one with phenyl boronic acid to obtain 3-bromo-5-(2-pyridyl)-1-phenyl-1,2-dihydropyridine-2-one; and
(c) reacting the 3-bromo-5-(2-pyridyl)-1-phenyl-1,2-dihydropyridine-2-one with 2-(2-cyanophenyl)-1,3,2-dioxaborinate to obtain perampanel.

2. The process according to claim 1, wherein the brominating agent is selected from N-bromosuccinimide (NBS), tribromoisocyanuric acid, acetic acid-bromine mixture, and liquid bromine.

3. The process according to claim 1, wherein the 3-bromo-5-(2-pyridyl)-1-phenyl-1,2-dihydropyridine-2-one is obtained by reacting 3-bromo-5-(2-pyridyl)-1,2-dihydropyridin-2-one and phenyl boronic acid in the presence of a copper salt and a base.

4. The process according to claim 1 wherein the copper salt is selected from copper acetate, copper bromide, and copper iodide, and a base is selected from an inorganic or an organic base.

5. The process according to claim 4, wherein the base is selected from sodium hydroxide, potassium hydroxide, lithium hydroxide, sodium carbonate, potassium carbonate, sodium bicarbonate, potassium bicarbonate, sodium hydride, potassium tert-butoxide, cesium carbonate, methyl lithium, butyl lithium, sodium amide, dialkyl lithium amide; triethylamine, diisopropyl ethyl amine, diethylamine, N-methyl morpholine, pyridine, piperidine, and DBU.

6. The process according to claim 1, wherein the 3-bromo-5-(2-pyridyl)-1-phenyl-1,2-dihydropyridine-2-one is reacted with 2-(2-cyanophenyl)-1,3,2-dioxaborinate in the presence of a palladium catalyst to obtain perampanel.

7. The process according to claim 6, wherein the palladium catalyst is tetrakis(tri-phenylphosphine)palladium.

8. A process for the preparation of 3-bromo-5-(2-pyridyl)-1-phenyl-1,2-dihydro-pyridine-2-one, the process comprising reacting 5-(2-pyridyl)-1,2-dihydropyridin-2-one with a brominating agent to obtain 3-bromo-5-(2-pyridyl)-1,2-dihydropyridin-2-one and reacting the 3-bromo-5-(2-pyridyl)-1,2-dihydropyridin-2-one with phenyl boronic acid to obtain 3-bromo-5-(2-pyridyl)-1-phenyl-1,2-dihydropyridine-2-one.

9. The process according to claim 8, wherein the brominating agent is selected from N-bromosuccinimide (NBS), tribromoisocyanuric acid, acetic acid-bromine mixture, and liquid bromine.

10. The process according to claim 8, wherein the 3-bromo-5-(2-pyridyl)-1-phenyl-1,2-dihydropyridine-2-one is obtained by reacting 3-bromo-5-(2-pyridyl)-1,2-dihydropyridin-2-one and phenyl boronic acid in the presence of copper acetate and pyridine.

11. The process according to claim 10, wherein the 3-bromo-5-(2-pyridyl)-1,2-dihydropyridin-2-one is obtained in 85% or more yield and having about 95% or more purity as measured by area percentage of HPLC.

12. 3-bromo-5-(2-pyridyl)-1,2-dihydropyridin-2-one having about 95% purity as measured by area percentage of HPLC.

13. Perampanel prepared by the process according to claim 1, having a purity of about 99% or more as measured by area percentage of HPLC.

14. A pharmaceutical composition comprising perampanel according to claim 13 and one or more pharmaceutically acceptable carriers, excipients or diluents.
